(19) United States
(12) Patent Application Publication
(10) Pub. No.: US 2002/0015662 A1
(43) Pub. Date: Feb. 7, 2002

(54) Inactivation of contaminants using photosensitizers and pulsed light

(76) Inventor: Dennis J. Hlavinka, Arvada, CO (US)

Correspondence Address: GAMBRO, INC, PATENT DEPARTMENT, 10810 W COLLINS AVE, LAKEWOOD, CO, 80215, US

(21) Appl. No.: 09/962,029

(22) Filed: Sep. 25, 2001

Related U.S. Application Data
(63) Continuation-in-part of application No. 09/596,429, filed on Jun. 15, 2000.
(60) Provisional application No. 60/235,999, filed on Sep. 27, 2000.

(51) Int. Cl. A61L 2/08

(52) U.S. Cl. 422/24; 422/28

(57) ABSTRACT

Methods and apparatuses are provided for inactivation of pathogens in fluids. Preferably the fluids contain blood or blood products and may include biologically active proteins. Preferred methods include the steps of adding an effective, non-toxic amount of a photosensitizer to a fluid and exposing the fluid to pulsed light, wherein the pulsed radiation emitting source emits radiation sufficient to activate the photosensitizer whereby pathogens are inactivated.

### CROSS-REFERENCE To RELATED APPLICATIONS

This Application is a continuation-in-part of U.S. application Ser. No. 09/596,429 filed Jun. 15, 2000, and U.S. application No. 60/235,999 filed Sep. 27, 2000, which are incorporated herein in their entirety to the extent not incompatible herewith.

### BACKGROUND

Contamination of blood products with infectious microorganisms such as HIV, hepatitis and other viruses and bacteria present a serious health hazard for those who must receive transfusions of whole blood or administration of various blood components such as platelets, red cells, blood plasma, Factor VIII, plasminogen, fibronectin, anti-thrombin III, cryoprecipitate, human plasma protein fraction, albumin, immune serum globulin, prothrombin complex plasma growth hormones, and other components isolated from blood. Blood screening procedures may miss pathogenic contaminants, and sterilization procedures which do not damage cellular blood components but effectively inactivate all infectious viruses and other microorganisms have not heretofore been available.

The term “blood product” as used herein includes all blood constituents or blood components and therapeutic protein compositions containing proteins derived from blood as described above. Fluids containing biologically active proteins other than those derived from blood may also be treated by the methods and devices of this invention.

The use of pathogen inactivating agents include certain photosensitizers, or compounds which absorb light of a defined wavelength and transfer the absorbed energy to an energy acceptor, have been proposed for inactivation of microorganisms found in blood products or fluids containing blood products. Such photosensitizers may be added to the fluid containing blood or blood products and irradiated.

The photosensitizers useful in this invention include any photosensitizers known to the art to be useful for inactivating microorganisms. A “photosensitizer” is defined as any compound which absorbs radiation of one or more defined wavelengths and subsequently utilizes the absorbed energy to carry out a chemical process. Examples of such photosensitizers include porphyrins, psoralens, dyes such as neutral red, methylene blue, acridine, toluidines, flavine (acriflavine hydrochloride) and phenothiazine derivatives, coumarins, quinolones, quinones, and anthroquinones. Photosensitizers of this invention may include compounds which preferentially adsorb to nucleic acids, thus focusing their photodynamic effect upon microorganisms and viruses with little or no effect upon accompanying cells or proteins. Other photosensitizers are also useful in this invention, such as those using singlet oxygen-dependent mechanisms. Most preferred are endogenous photosensitizers. The term “endogenous” means naturally found in a human or mammalian body, either as a result of synthesis by the body or because of ingestion as an essential foodstuff (e.g. vitamins) or formation of metabolites and/or byproducts in vivo. Examples of such endogenous photosensitizers are alloxazines such as 7,8-dimethyl-10-ribityl isoalloxazine (riboflavin), 7,8,10-trimethylisoalloxazine (lumiflavin), 7,8-dimethylalloxazine (lumichrome), isoalloxazine-adenine dinucleotide (flavine adenine dinucleotide [FAD]), alloxazine mononucleotide (also known as flavine mononucleotide [FMN] and riboflavine-5-phosphate), vitamin Ks, vitamin L, their metabolites and precursors, and napththoquinones, naphthalenes, naphthols and their derivatives having planar molecular conformations. The term “alloxazine” includes isoalloxazines. Endogenously-based derivative photosensitizers include synthetically derived analogs and homologs of endogenous photosensitizers which may have or lack lower (1-5) alkyl or halogen substituents of the photosensitizers from which they are derived, and which preserve the function and substantial non-toxicity thereof. When endogenous photosensitizers are used, particularly when such photosensitizers are not inherently toxic or do not yield toxic photoproducts after photoradiation, no removal or purification step is required after decontamination, and a treated product can be directly returned to a patient's body or administered to a patient in need of its therapeutic effect without any further required processing. Using endogenous photosensitizers to inactivate pathogens in a blood product are described in U.S. Pat. No. 6,258,577 and No. 6,277,337, herein incorporated by reference.

Non-endogenous photosensitizers based on endogenous structures, such as those described in U.S. Pat. No. 6,268,120, may also be incorporated by reference herein. These non-endogenous photosensitizers and endogenously-based derivative photosentizers may be referred to herein as endogenously-based derivative photosensitizers.

One mechanism by which these photosensitizers may inactivate pathogens is by interfering with nucleic acids, so as to prevent replication of the nucleic acid. As used herein, the term “inactivation of a pathogen” means totally or partially preventing the pathogen from replicating, either by killing the pathogen or otherwise interfering with its ability to reproduce. Specificity of action of the photosensitizer is conferred by the close proximity of the photosensitizer to the nucleic acid of the pathogen and this may result from binding of the photosensitizer to the nucleic acid. “Nucleic acid” includes ribonucleic acid (RNA) and deoxyribonucleic acid (DNA).

Microorganisms or pathogens which may be eradicated or inactivated using pathogen inactivation agents or photosensitizers include, but are not limited to, viruses (both extracellular and intracellular), bacteria, bacteriophages, fungi, blood-transmitted parasites, and protozoa. Exemplary viruses include acquired immunodeficiency (HIV) virus, hepatitis A, B and C viruses, sinbis virus, cytomegalovirus, vesicular stomatitis virus, herpes simplex viruses, e.g. types I and II, human T-lymphotropic retroviruses, HTLV-III, lymphadenopathy virus LAV/IDAV, parvovirus, transfusion-transmitted (TT) virus, Epstein-Barr virus, and others known to the art. Bacteriophages include ΦX174, Φ6, λ, R17, T₄, and T₂. Exemplary bacteria include P. aeruginosa, S. aureus, S. epidermis, L. monocytogenes, E. coli, K. pneumonia and S. marcescens.

The fluid to be pathogen inactivated has the photosensitizer added thereto, and then the resulting fluid mixture may be exposed to photoradiation of the appropriate wavelength and amount to activate the photosensitizer, but less than that which would cause non-specific damage to the biological components or substantially interfere with biological activity of other proteins present in the fluid. The wavelengths of photoradiation used may depend on the type of photosensitizer selected and the composition of the fluid, as is known in the art.

A number of systems and methods for irradiating pathogens in a fluid with light are known in the art. For example, U.S. Pat. No. 5,762,867 is directed toward a system for activating a photoactive agent present in a body fluid with light emitting diodes (LEDs). U.S. Pat. No. 5,527,704 is directed toward an apparatus containing LEDs used to activate a fluid containing methylene blue. U.S. Pat. No. 4,880,512 uses pulsed lasers to photolyse nucleic acids contained in a fluid. None of these above-mentioned patents uses pulsed light in combination with a photsensitizer to inactivate pathogens in a fluid containing pathogens.

The term light source or radiation source as defined herein means an emitter of radiant energy, and may include energy in the radio, infrared, visible, ultraviolet, X-ray and/or gamma spectra.

The photosensitizer may be added directly to the fluid to be pathogen inactivated, or may be flowed into the photopermeable container separately from the fluid being treated, or may be added to the fluid prior to placing the fluid in the photopermeable treatment container. The photosensitizer may also be added to the photopermeable container either before or after sterilization of the treatment container.

The fluid containing the photosensitizer may also be flowed into and through a photopermeable container for irradiation, using a flow through type system. Alternatively, the fluid to be treated may be placed in a photopermeable container which is agitated and exposed to photoradiation for a time sufficient to substantially inactivate the microorganisms, in a batch-wise type system.

The term “container” refers to a closed or open space, which may be made of rigid or flexible material, e.g., may be a bag or box or trough. In one embodiment, the container may be closed or open at the top and may have openings at both ends, e.g., may be a tube or tubing, to allow for flow-through of fluid therein. A cuvette has been used to exemplify one embodiment of the invention involving a flow-through system. Collection bags, such as those used with the Trima® and/or Spectra™ apheresis systems of Gambro, Inc., (f/k/a Cobe Laboratories, Inc., Lakewood, Colo., USA), have been used to exemplify another embodiment involving a batch-wise treatment of the fluid.

The term “photopermeable” means the material of the treatment container is adequately transparent to photoradiation of the proper wavelength for activating the photosensitizer. In a flow-through system, the container has a depth (dimension measured in the direction of the radiation from the photoradiation source) sufficient to allow photoradiation to adequately penetrate the container to contact photosensitizer molecules at all distances from the light source and ensure inactivation of pathogens in the fluid to be decontaminated, and a length (dimension in the direction of fluid flow) sufficient to ensure a sufficient exposure time of the fluid to the photoradiation. The materials for making such containers, the depths and lengths of containers may be easily determined by those skilled in the art, and together with the flow rate of fluid through the container, the intensity of the photoradiation and the absorptivities of the fluid components, e.g., plasma, platelets, red blood cells, will determine the amount of time the fluid should be exposed to photoradiation.

After treatment, the blood or blood product may be stored for later delivery to a patient, concentrated, infused directly into a patient or otherwise processed for its ultimate use.

### SUMMARY

The present invention provides a method and apparatus for irradiating a fluid containing pathogens and a photoactive agent with pulsed light of an appropriate wavelength, to activate the photoactive agent contained therein and thereby inactivate any pathogens contained in the product.

The present invention involves using a pulsed light source to generate the photoradiation to which the fluid is exposed. The pulsing of the light may allow higher intensities of light to be used than may otherwise be used without causing damage to either the blood product being irradiated or to the light sources due to the short duration of the radiation exposure.

Alternative sources of radiation may be used, depending on a variety of factors, including, but not limited to the wavelength desired and the power at the desired wavelength. LEDs (light emitting diodes) which pulse the light as it is emitted may also be used in the described invention.

The lights may be configured in many different ways, depending upon the particular apparatus being used.

One embodiment of a radiation or treatment chamber useful with the methods of the present invention includes a bank or banks or arrays of lights. Such lights may produce a particular wavelength of light that corresponds to the particular wavelength preferred for the type of fluid being treated. In another embodiment, the banks or arrays of lights may be interchangeable. For example, in one apparatus, one bank of lights that produces a wavelength suitable for irradiating platelets may be interchanged with another bank of lights that produces a wavelength suitable for irradiating red blood cells.

A radiation enhancer such as a second radiation source or a reflective surface may be included in the radiation or treatment chamber. The radiation enhancer may be placed adjacent to the container containing the fluid to be irradiated or opposite the radiation source to increase the amount of radiation contacting the fluid within the container.

The radiation or treatment chamber may also preferably include a means for producing movement in the fluid to be irradiated. Movement provides many benefits including improving the efficiency of the inactivation reactions by helping mix the photosensitizer with the fluid to be photoinactivated to provide turnover of the fluid within the container at the container-light interface.

Positioning the fluid to be inactivated so that it receives energy of sufficient wavelength and power to induce inactivation of pathogens contained in the fluid may include a support platform, a shelf or a tray for the sample to be disposed upon; an opening or gap between two supports which may be a light or light arrays, where the fluid within the container is positioned between the supports; or other means known in the art. The support platform may move, as in a conveyer line, or may oscillate or agitate. The fluid-holding shelves may be transparent to one or more of the wavelengths of light applied. The fluid within the container may also be placed on a support surface between two or more sources of radiation, like a sandwich.

### BRIEF DESCRIPTION OF THE FIGURES

FIG. 1 is a cross-sectional view of a treatment chamber which may be used in the present invention.

FIG. 2 is a cross-sectional view of a treatment chamber like that of FIG. 1, but with an alternative reflective surface that may also be used in the present invention.

FIG. 3 is a plan view of an array of LEDs that may be used in the present invention.

FIG. 4 is a schematic diagram of one type of LED circuit that may be used with the present invention.

FIG. 5a is a schematic view of the light pulses produced by a LED irradiating a container according to the present invention.

FIG. 5b is a schematic view of the light pulses produced by an incandescent light bulb irradiating a container according to the present invention.

FIG. 6 is another embodiment of a treatment chamber which may be used in the present invention.

### DETAILED DESCRIPTION

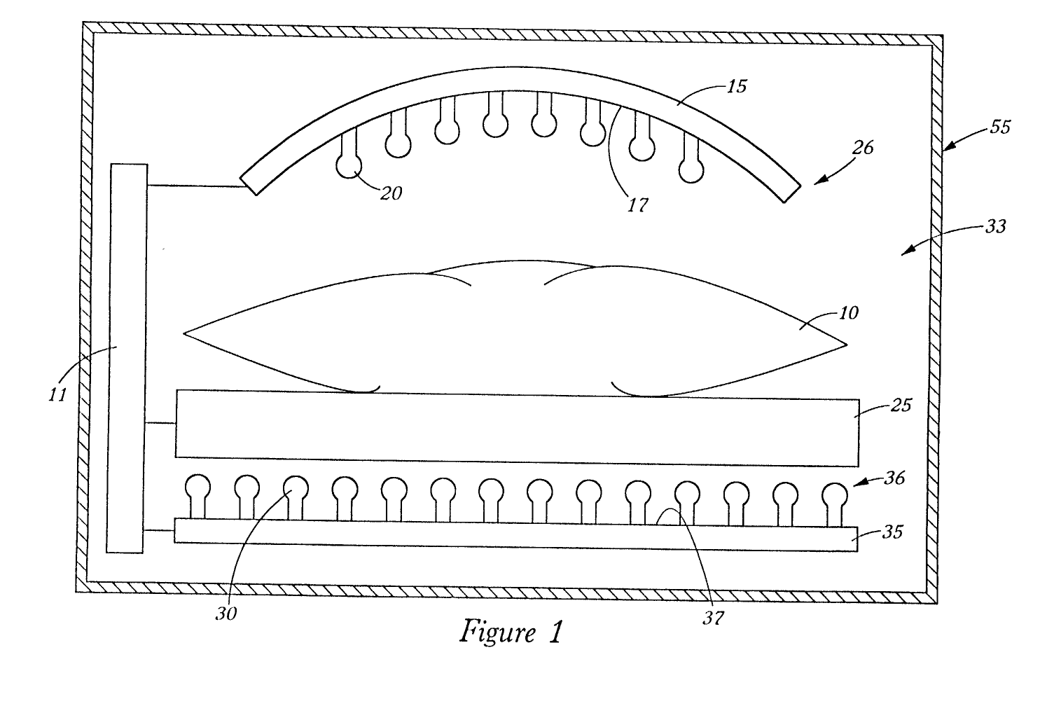

FIG. 1 shows in a cross-sectional view, the inside of a radiation or treatment chamber of one type of apparatus that may be used in the present invention. It should be noted that throughout the description of the invention, like elements have been given like numerals. The apparatus 55, used for inactivating a fluid which may contain pathogens, consists of an internal chamber 33 having at least one source of radiation 26. In one preferred embodiment, the internal chamber may contain a second source of radiation 36. Each radiation source 26 and 36 respectively, is depicted as including a plurality of discrete radiation-emitting elements. The internal chamber 33 further consists of a support platform 25 for supporting the fluid container 10 containing the fluid to be irradiated, and a control unit 11.

As introduced above, two sources of radiation are shown within internal chamber 33. Radiation source 26 may be located along the top portion of the internal chamber 33 above the container 10 which holds or contains the fluid to be irradiated, while radiation source 36 may be located along the bottom portion of the internal chamber 33 below the container 10. Although not shown, radiation sources may also be located along some or all of the sides of the internal chamber 33 perpendicular to the container 10. The radiation or treatment chamber 55 may alternatively contain a single radiation source at any location within the internal chamber 33 and still comply with the spirit and scope of the present invention.

As shown in FIG. 1, the radiation source including a plurality of radiation-emitting elements collectively designated as source 26 includes an upper support substrate 15 containing a plurality of discrete radiation emitting elements or discrete light sources (see discrete source 20 as one example) mounted thereon. The support substrate 15 may be in an arcuate shape as shown, in a flat shape, or in other configurations which are not shown. Thus, the upper support substrate 15 could also be in a shape other than arcuate without departing from the spirit and scope of the invention.

As further depicted in FIG. 1, the radiation source collectively designated as discrete source 36 includes a lower support substrate 35 which also contains a plurality of discrete radiation emitting elements or discrete light sources (see discrete source 30 as another example). Lower support substrate 35 preferably runs parallel to support platform 25. The lower support substrate 35 may be substantially flat as shown, or may be in an arcuate shape similar to element 15 above, or may be in a shape other than arcuate, without departing from the spirit and scope of the invention.

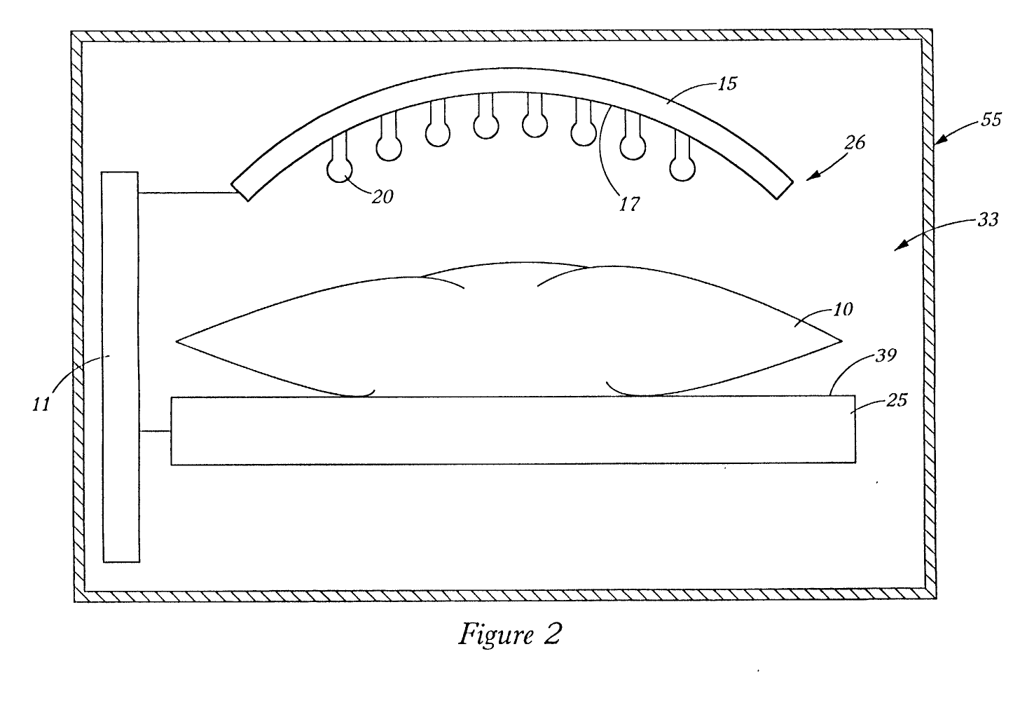

The support substrates 15 and 35 may include at least one reflective surface, and as shown, may include two or more reflective surfaces 19 and 37 thereon. Reflective surface 17 is shown as running contiguous with upper support substrate 15. Reflective surface 37 is shown as running contiguous with lower support substrate 35. The reflective surfaces 17 and 37 may also run contiguously with only a portion of support substrates 15 and 35. As shown in FIG. 1, discrete light source devices 20 and 30 extend outwardly away from the surface of the support substrates 15 and 35. Alternatively, a discrete light source could be recessed into the surface such that the surface surrounds each discrete light source in a parabolic shape (not shown). The support substrate may or may not have reflective surfaces. Most preferably, the reflective surface may not contain any light sources. Such a reflective surface containing no light sources (not shown) may be located within the treatment chamber on a side opposite from the radiation source. As shown in FIG. 2, the support platform 25 may have a reflective surface 39. This reflective surface 39 on support platform 25 may be in place of, or may be in addition to another reflective surface (see element 17 as one example) within the treatment chamber. There may also be no reflective surfaces at all within the treatment chamber.

In any of these reflective surface embodiments, the reflective surface may be coated with a highly reflective material which serves to reflect the radiation pulses emitted from the lights back and forth throughout the treatment chamber until the pulse is preferably completely absorbed by the fluid being irradiated. The highly reflective nature of the reflective surface reflects the emitted light back at the fluid-filled bag or container 10 with minimum reduction in the light intensity.

As shown in FIG. 1, support platform 25 is positioned within the internal treatment chamber 3. The support platform 25 may be located substantially in the center of the radiation or treatment chamber (as shown in FIG. 1), or may be located closer to either the top portion or the bottom portion of the treatment chamber without departing from the spirit and scope of the present invention. The support platform 25 supports the container 10 containing the fluid to be irradiated. The support platform 25 may also be defined as a tray or a shelf. Additionally or alternatively, the platform 25 may be made of a photopermeable material to enable radiation emitted by the pulsed lights to be transmitted through the platform and penetrate the fluid contained within the container 10. The platform may also be a wire or other similar mesh-like material to allow maximum light transmissivity therethrough. The support platform 25 is preferably capable of movement in multiple directions within the treatment chamber. An agitator, such as a Helmer flatbed agitation system available from Helmer Corp. (Noblesville, Ind., USA) may also be used. This type of agitator provides oscillary motion. Other types of agitators may also be used to provide motion to the fluid contained within the container 10, without departing from the spirit and scope of the invention. The support platform 25 may alternatively rotate in multiple possible directions within the radiation chamber in varying degrees from between 0° to 360°. The support platform 25 may also oscillate back and forth, or side to side along the same plane. As a further alternative, one or more of the light sources may also move in a coordinated manner with the movement of the support platform. Such oscillation or rotation would enable the majority of the photosensitizer and fluid contained within the container 10 to be exposed to the light emitted from each of the discrete radiation sources (e.g. discrete sources 20 and 30), by continually replacing the exposed fluid at the light-fluid interface with fluid from other parts of the bag not yet exposed to the light pulses. Such mixing brings to the surface new fluid to be exposed to light with each new pulse of radiation.

The movement of both the support platform 25 and/or the radiation sources 26 and 36 may be controlled by control unit 11. The control unit 11 may control the rate of light emission in either a continuous or a pulsed mode, and the control unit 11 may be provided with a selector switch (not shown) in order to select which mode is to be employed. In the “continuous” mode the radiation source emits radiation in a continuous manner. In the “pulsed” mode the radiation source emits pulses of radiation.

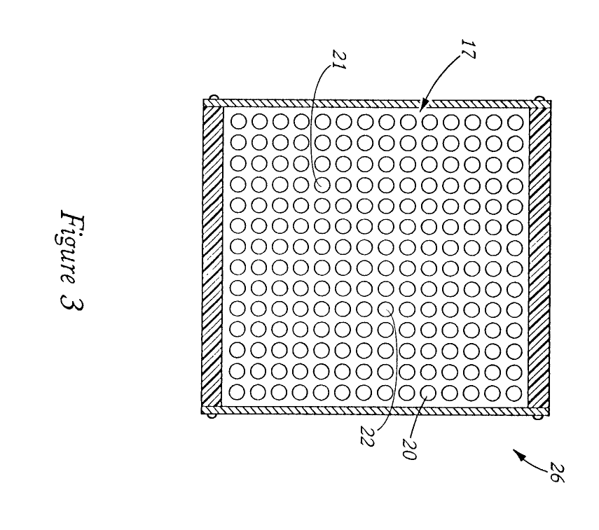

As shown in FIG. 3, each radiation source 26 may consist of a bank or array of a plurality of discrete LEDs devices. LED devices 20, 21 and 22 are self-contained emitters of radiation. Each LED emits a single color of light when an electrical current is applied.

Each of the LED devices in the array 26 may emit the same wavelengths of light, or because each source is discrete, each source may emit particular wavelengths of radiation independently of the emission of radiation by any other light source devices also contained within the light array. For example, LED device 20 may emit light of different wavelengths than the wavelengths emitted by LED device 22. This could be useful in providing different wavelengths of light for different blood products, as may be desired.

The discrete radiation sources or lights may be arranged in banks or arrays containing multiple rows of individual lights, or may be arranged in a single row (not shown). As shown in FIG. 3, if LED devices are used, a plurality of discrete LED devices may be arranged in multiple rows. The lights may also be staggered or offset from each other (not shown). If a bank or an array of LED lights is located in both the top and the bottom of the irradiation chamber 55 (see FIG. 1), each bag or container 10 containing fluid to be irradiated will be exposed to light pulses on both the top and the bottom surfaces of the bag 10. A reflective surface 17 (like that shown in FIG. 1) may also be part of the array.

As described, the lights used in this invention may be LED devices. LED devices are available from any one of a number of companies. Some companies that manufacture LED devices useful in this invention are Cree, Inc. (Durham, N.C., USA); Nichia, Co. (Tokushima, JP); Kingbright, Corp. (City of Industry, Calif., USA) and Lumileds Lighting, LLC (San Jose, Calif., USA).

The light devices 20, 21, and 22 may be placed in an interchangeable format which would allow one set of LED devices to be removed and replaced by another set of LED devices. For example, depending upon the type of blood product to be irradiated, the LED devices which emit particular wavelengths of light that correspond to the type of blood product being irradiated may be easily inserted into the irradiation chamber. As one example, one array containing a radiation source which corresponds to a first type of blood product is inserted into the treatment chamber. After irradiation of the first blood product, the array may be removed from the treatment chamber. If a second type of blood product needs to be treated, a second array corresponding to the second type of blood product is inserted into the treatment chamber. After irradiation of the second blood product the second array may be removed from the treatment chamber.

Alternatively, as introduced above, a single bank of LED devices may contain multiple discrete light sources which each emit a variety of different wavelengths for this purpose as well. Control unit 11 (see FIG. 1) may be used to selectively engage the particular discrete LED sources which emit light pulses of particular wavelengths, depending upon the type fluid to be irradiated. These concepts may be used with non-LED light devices as well.

The radiation sources used in this invention are most preferably capable of being pulsed. In some embodiments, pulsed radiation sources may be generated by intense flashes from an inert gas such as xenon and each pulse can last less than one millisecond in duration.

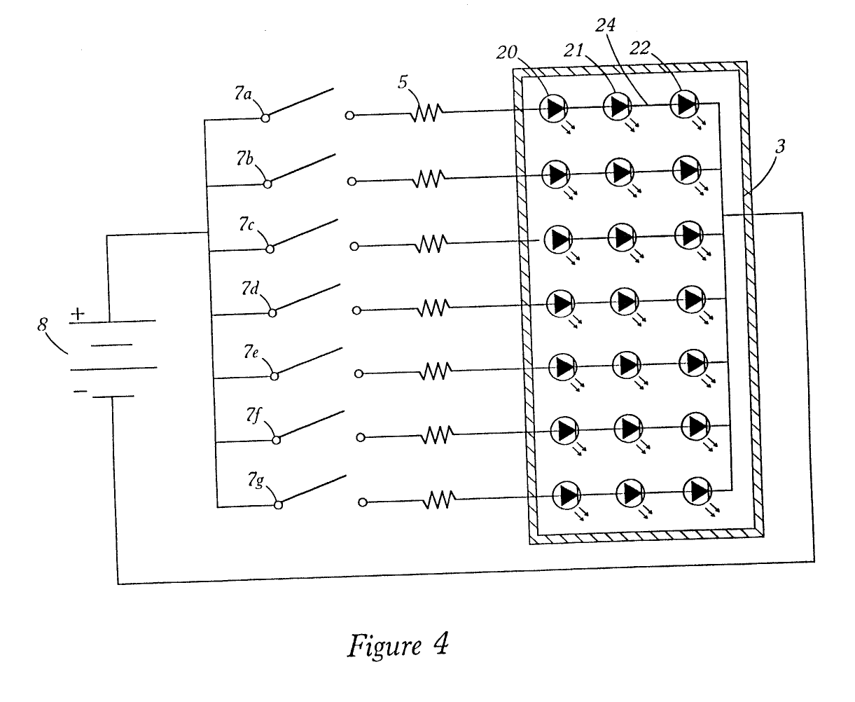

In an alternative system using LEDs capable of being pulsed, one method of generating light pulses may be by a series of switches such as those shown schematically in FIG. 4. FIG. 4 shows one of many possible schematic LED circuits which may be used in this invention. Each discrete radiation source (see LED devices 20, 21 and 22 as examples) is arranged in the form of a LED matrix 3 comprising a plurality of parallel branches (see branch 24 as one example). Each branch, such as branch 24 contains a predetermined number of discrete LED devices (see devices 20, 21 and 22) preferably in series. For example, LED devices 20, 21 and 22 may be connected to one another in series and together constitute one parallel branch 24 of the diode matrix 3. The current flowing through each branch may be limited by means of a series resistor (element 5 for one such example). Each branch may be connected to one of a plurality of switches (see switches 7a-g) which are capable of being opened and closed. When switch 7a (as one such example) is in a closed position (not shown) current is able to flow from the voltage source 8, through switch 7a, through the resistor 5, to each discrete LED 20, 21 and 22 located within branch 24. When switch 7a is open as shown in FIG. 4, no current is able to flow down that particular branch to light each discrete LED contained within this branch. The control unit (see element 11 in FIG. 1) may be connected to any one or more of switches 7a-g. Each switch may thereby be controlled individually, or in coordination with one or more of the other switches in the matrix 3 to provide current to the desired branch or branches. Determination of the switching sequence may depend on the type and/or duration of the pulse wanted, which in turn may be determined by the type of fluid being irradiated. In another embodiment (not shown) a varistor may be used in place of any one or more of the resistors (see element 5 as an example). The varistor may alternatively be used to enable the LEDs to be pulsed on or off, as well as to brighten and/or dim the light intensity. Any one of a number of commonly used switching mechanisms known in the art may be used in providing the desired pulsing of this invention.

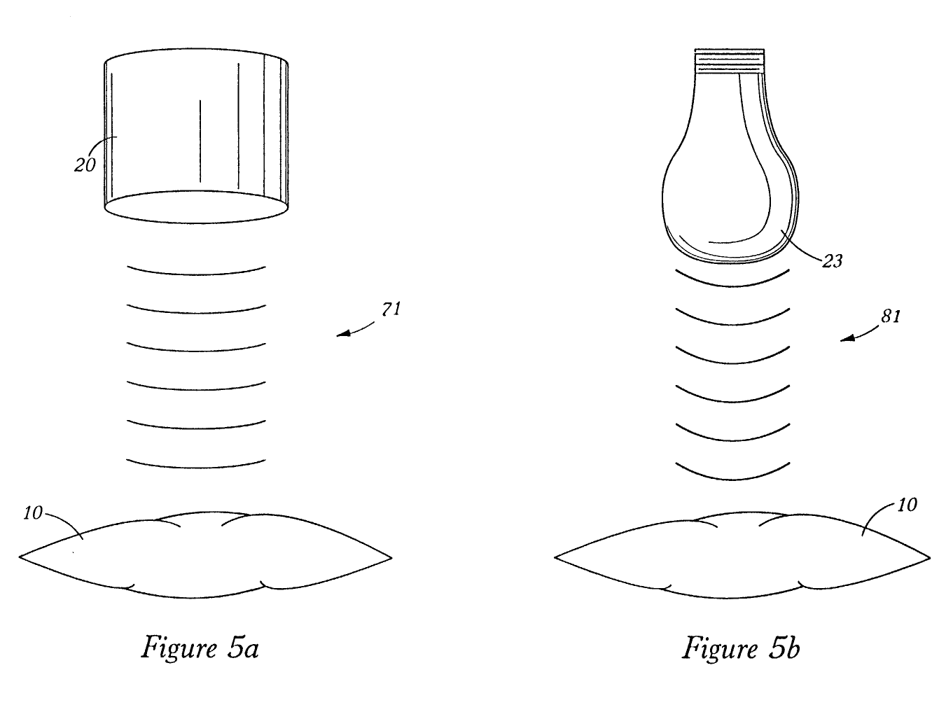

FIGS. 5a and 5b show alternative cross-sectional views of light pulses possibly produced by different types of light sources. FIG. 5a shows a LED 20 emitting light pulses 71 which irradiate bag 10. The light pulses could be in the visible or the UV spectrum or both, depending upon the type of fluid being irradiated in bag 10. FIG. 5b shows an incandescent light source 23 which may also emit pulses of light 81 in the desired wavelength for the particular product being irradiated.

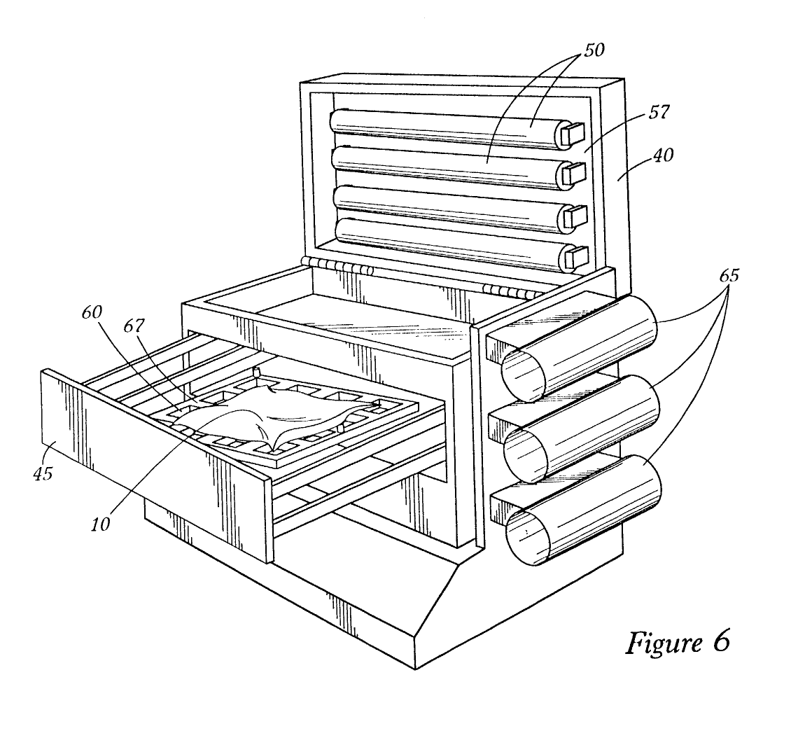

FIG. 6 shows an alternative embodiment of an irradiation or treatment chamber to be used with the present invention. A bank of pulsed light sources 50 may be located within the top of the irradiation chamber extending from lid 40. A reflective surface 57 is shown as part of the inner surface of lid 40, however, reflective surface 57 or another one or more surfaces (not shown) may be located anywhere within the radiation chamber as introduced above.

The lid 40 is capable of being opened and closed. During exposure of the bag 10 containing the fluid to be irradiated to the light sources, the lid 40 is in a closed position (not shown). To add or remove the bag 10 containing the fluid to be irradiated from the irradiation chamber, a drawer 45 located on the front of the irradiation chamber may be disposed in an open position (as shown). During the irradiation procedure, the drawer 45 is placed in a closed position (not shown).

The pulsed light sources 50 may be fluorescent or incandescent tubes, which stretch the length of the irradiation chamber, or may be a single light source which extends the length and width of the entire chamber (not shown). The LEDs shown in FIG. 3 may also be used in this embodiment. Although lights are shown as being located only on the top of the irradiation chamber, lights may also be located in the bottom of the chamber as well. Some examples of light sources that may be used include those available from the Xenon Corporation (Woburn, Mass., USA) or from PurePulse Technologies, Inc. (San Diego, Calif., USA) among other companies.

As shown in FIG. 6, the support platform 67 may be located within and/or forming part of drawer 45. The support platform 67 may contain gaps 60 or holes or spaces within the platform 67 to allow radiation to penetrate through the gaps directly into the container 10 containing fluid to be irradiated.

Although pulsed light systems may generally maintain the system (light sources and container) at cooler temperatures, a cooling system may also optionally be included. As shown in FIG. 6, air cooling using at least one fan 65 may be preferred but it is understood that other well-known systems can also be used. Although not shown in FIG. 6, the method may also include the use of temperature sensors and other cooling mechanisms where necessary to keep the temperature below temperatures at which desired proteins and blood components in the fluid being irradiated are damaged. Preferably, the temperature is kept between about 0° C. and about 45° C., more preferably between about 4° C. and about 37° C., and most preferably about 28° C.

The pulsed light used may be in the visible spectrum, the ultraviolet spectrum, or both the visible and ultraviolet spectra. Any suitable wavelength or wavelengths of light may be used in any proportion and amount of energy that produces the desired level of inactivation of pathogens in the fluid being irradiated. As one example, half the light emitted may be in the visible spectrum and about half the light emitted may be in the ultraviolet spectrum. As another alternative, not meant to be limiting, about one-third of light may be in one spectral range and the other two-thirds of light in a different spectral range.

In one embodiment of the invention, multiple light sources (or multiple arrays of light sources) may be used to provide multiple wavelengths of light.

The pulsed light sources may also be selected so that each discrete radiation source or device has either a broad band or narrow band spectrum. Broad band pulsed light sources include light in both the visible and ultraviolet range and/or in the infrared wavelength range. Thus, a single light source can provide a mixture of both visible and ultraviolet light. If ultraviolet light is used, the amount of ultraviolet light is preferably kept to a level that minimizes non-specific damage to desired fluid components. Generally, this may be provided by using 50% or less ultraviolet light relative to the total light energy delivered. Non-specific damage is damage that damages components contained within the fluid being irradiated. A narrow band pulsed light source is preferably used with respect to irradiation of the selected blood components.

The photoradiation in both the ultraviolet and visible spectra may be supplied concurrently or sequentially, with the visible portion preferably being supplied first. As described above, the photoradiation source may be a simple lamp or may consist of multiple lamps radiating at differing wavelengths. The photoradiation sources may also consist of multiple discrete LED devices radiating at the same or different wavelengths. Individual LED devices which emit different wavelengths of light and pulse in differing frequencies may be combined together in one array. Alternatively, one array could contain multiple LED devices which all emit light in the same wavelength range.

The particular parameters of each pulsed light source can be selected to provide irradiation of sufficient intensity to activate the photosensitizer in a pathogen inactivation process without damage to the blood or blood components being irradiated. The photoradiation source should be capable of delivering a sufficient amount of light to activate the photosensitizer, preferably from about 3 to at least about 200 J/cm². The preferred range of wavelengths of light supplied in pulsed light fashion in accordance with the present invention are between 280-550 nm. However, the preferred wavelengths of light used may depend upon the type of photosensitizer used. LEDs or other types of lights which emit pulses of light having wavelengths of around 400-550 nm may be used for irradiating platelets, LEDs or other types of lights which emit pulses of light emitting wavelengths of around 260-500 nm may be used for irradiating plasma, and LEDs or other types of lights emitting pulses of light having wavelengths of around 430-550 nm may be used for irradiating RBCs.

The pulsed light sources may emit pulses of radiation at pulse rates of between one pulse per 0.25 seconds up to one pulse per 100 seconds. Each individual pulse may last between 0.1 and 10 seconds.

The pulsed light sources produce their high-intensity, short duration output pulses in a repetitive fashion. To insure that all of the fluid to be irradiated is subjected to substantially the same irradiation conditions, each discrete light device should produce the same amount of repetitions, and the bag should be placed in a location which exposes the greatest possible surface of the bag to radiation.

Pulsed irradiation using very high energy, short duration pulses may have advantages over lower energy continuous irradiation. The pulsing of the light source may allow for a higher intensity than could otherwise be used without damage to either the blood product or to the light sources. The high energy pulses may also penetrate deeper into the fluid being irradiated. This would likely enhance pathogen inactivation, especially in more opaque blood products such as red blood cells or plasma containing high levels of lipids. In more opaque solutions, mixing is enhanced because the thicker layers of solution photolyzed by the high intensity flash are turned over more readily. With low intensity light, thinner layers of photolyzed solution must be turned over, and this is more difficult to achieve in a system being mixed.

Pulsing the light instead of providing continuous light emission is beneficial for several reasons. The intensity of light which may be emitted from the radiation sources may be increased dramatically if the lights are allowed to be turned off and rested in between light pulses. As stated above, pulsing the light at a high intensity also allows for greater depth of light penetration into the fluid being irradiated, thus irradiating a thicker layer of fluid with each pulse.

Synchronizing the radiation sources to pulse in a coordinated manner with the movement of the support platform allows for fluid which has already been irradiated to be replaced by fluid which has not been irradiated. One such sequence may be as follows. The radiation sources are pulsed on for a period of time, perhaps for between one and ten seconds. The radiation sources are then pulsed off for another period of time, perhaps again for between one and ten seconds. While the radiation source is off, the support platform moves in a manner as described above, to replace the fluid which had been exposed to radiation with the previous pulse, with fluid which has not yet been exposed to radiation. When the radiation sources are then pulsed on again, new, unexposed fluid is now exposed to the radiation sources.

Synchronizing pulses to mixing cycles may also optimize the inactivation process. If more than one bank or array of pulsing lights are used, each bank or array may be programmed to pulse simultaneously with the other banks of lights, or may be programmed to pulse the lights independently of the other banks of lights.

Pulsed light sources may also allow for adjustment of the duration and intensity of light energy per pulse to assure optimum eradication of the viruses or pathogens with the least amount of exposure of the blood product to light. The lights may be pulsed in a frequency corresponding to the rocking of the support platform, as set forth above. Alternatively, the lights may not pulse completely on and then pulse completely off. The lights may pulse in an increasing or decreasing frequency of intensity. For example, the lights could dim in brightness and then could increase in brightness or intensity. The lights may also pulse in differing cycles or frequencies depending on the type of fluid being inactivated.

It should be understood that various changes and modifications to the presently preferred embodiments described herein will be apparent to those skilled in the art. Such changes and modifications can be made without departing from the spirit and scope of the present invention and without diminishing its attendant advantages.

Accordingly, what is claimed is:

1. A method for inactivating pathogens in a blood product comprising
adding a photosensitizer to the blood product to form a mixture;
exposing the mixture of the blood product and the photosensitizer to light; and
pulsing the light while exposing the mixture to the light.

2. The method of claim 1 wherein the photosensitizer is an endogenous photosensitizer.

3. The method of claim 1 wherein the photosensitizer is an isoalloxazine.

4. The method of claim 1 wherein the photosensitizer is riboflavin.

5. The method of claim 1 wherein the step of pulsing the light includes pulsing the light within the range of 280-550 nm.

6. The method of claim 1 wherein the step of pulsing the light includes pulsing the light within the wavelength range corresponding to the type of blood component being irradiated.

7. A treatment chamber for inactivating pathogens in a fluid containing a photosensitizer comprising
at least one pulsed radiation emitting source;
a support platform for holding the fluid to be irradiated; and
a control unit for controlling the pulsed light emitting source.

8. The treatment chamber of claim 7 wherein the support platform is capable of movement in multiple directions within the treatment chamber.

9. The treatment chamber of claim 7 wherein the control unit further controls the movement of the support platform.

10. The treatment chamber of claim 7 wherein the support platform is made of photopermeable material.

11. The treatment chamber of claim 7 wherein the treatment chamber further comprises at least one reflective surface.

12. The treatment chamber of claim 7 wherein the support platform is made of a reflective surface.

13. The treatment chamber of claim 7 wherein the pulsed radiation emitting source further comprises an array containing a plurality of discrete lights.

14. The treatment chamber of claim 13 wherein the array containing a plurality of discrete lights further comprises a plurality of LEDs.

15. The treatment chamber of claim 13 wherein the array containing a plurality of discrete lights further comprises a plurality of incandescent lights.

16. The treatment chamber of claim 7 wherein the pulsed radiation emitting source further comprises fluorescent tubes.

17. The treatment chamber of claim 7 wherein the pulsed radiation emitting source further comprises a single pulsed light source extending the length and width of the treatment chamber.

18. The treatment chamber of claim 7 wherein the pulsed radiation emitting source emits radiation in the visible spectrum.

19. The treatment chamber of claim 7 wherein the pulsed radiation emitting source emits radiation in the ultraviolet spectrum.

20. The treatment chamber of claim 7 wherein the pulsed radiation emitting source emits radiation in both the visible and the ultraviolet spectrum.

21. The treatment chamber of claim 7 wherein the pulsed radiation emitting source is adapted to be removed and replaced with another pulsed radiation emitting source.

22. The treatment chamber of claim 7 wherein the pulsed radiation emitting source emits wavelengths of light in the range of between 280 nm and 550 nm.

23. The treatment chamber of claim 7 wherein the pulsed radiation emitting source emits pulses of radiation which last between 0.1 and 10 seconds.

24. The treatment chamber of claim 7 wherein the pulsed radiation emitting source emits pulses of radiation at pulse rates of between one pulse per 0.25 seconds and one pulse per 100 seconds.

25. The treatment chamber of claim 7 wherein the control unit controls the pulsed radiation emitting source in coordination with the movement of the support platform.

26. The treatment chamber of claim 7 wherein the pulsed radiation emitting source further comprises a plurality of discrete light sources which each emit light of different wavelengths.

27. The treatment chamber of claim 7 wherein the pulsed radiation emitting source further comprises a plurality of discrete light sources which each emit light of similar wavelengths.

28. The treatment chamber of claim 13 wherein the control unit controls the discrete radiation emitting source wherein such sources emit light in different wavelength ranges.

29. A method of irradiating a blood product comprising the steps of:
adding an amount of photosensitizer necessary to inactivate any pathogens contained in the blood product;
exposing the blood product and photosensitizer to pulsed light; and
mixing the blood product and photosensitizer during the exposing step to expose the majority of the fluid to the light pulses.

30. A method of irradiating a blood product contained within a photopermeable bag comprising the steps of:
adding riboflavin to the bag in an amount necessary to inactivate any pathogens contained in the blood product;
exposing the bag containing at least the blood product and riboflavin to pulsed light; and
mixing the contents of the bag during the exposing step.

31. A method of inactivating pathogens contained in different components of blood in a single treatment chamber, the method comprising
determining a first type of blood component to be treated in the single treatment chamber;
inserting a first array containing a radiation emitting source which corresponds to the first type of blood component to be treated;
irradiating the first type of blood component;
removing the first array corresponding to the first type of blood component to be treated;
determining a second type of blood component to be treated;
inserting a second array containing a radiation emitting source which corresponds to the second type of blood component to be treated; and
irradiating the second type of blood component.

32. A method of irradiating a blood product comprising the steps of:
a) adding an amount of photosensitizer necessary to inactivate any pathogens contained in the blood product;
b) pulsing a radiation source on to expose the blood product and photosensitizer to radiation;
c) pulsing the radiation source off to stop exposure of the blood product and photosensitizer to radiation;
d) mixing the blood product and photosensitizer during the step of pulsing the radiation source off; and
e) repeating steps b), c) and d).
